(19) United States
(12) Patent Application Publication
(10) Pub. No.: US 2016/0029985 A1
(43) Pub. Date: Feb. 4, 2016

(54) X-RAY COMPUTED TOMOGRAPHY APPARATUS

(71) Applicants: Kabushiki Kaisha Toshiba, Minato-ku (JP); Toshiba Medical Systems Corporation, Otawara-shi (JP)

(72) Inventors: Tooru KATO, Nasushiobara (JP); Hiroaki Nakai, Nasushiobara (JP); Mikihito Hayashi, Otawara (JP)

(73) Assignees: Kabushiki Kaisha Toshiba, Minato-ku (JP); Toshiba Medical Systems Corporation, Otawara-shi (JP)

(21) Appl. No.: 14/884,119

(22) Filed: Oct. 15, 2015

Related U.S. Application Data
(63) Continuation of application No. PCT/JP2014/060959, filed on Apr. 17, 2014.

(30) Foreign Application Priority Data
Apr. 18, 2013 (JP) 2013-087765

(51) Int. Cl. A61B 6/00; A61B 6/03

(52) U.S. Cl. CPC A61B 6/4488; A61B 6/035; A61B 6/5205; A61B 6/586

(57) ABSTRACT

To solve the problems described above, an X-ray computed tomography apparatus includes an X-ray tube, a scintillator, a photoelectric convertor, a thermal storage material, a rotating portion, a rotating mechanism, and image generating circuitry. The X-ray tube generates an X-ray. The scintillator converts the X-ray generated by the X-ray tube into light. The photoelectric convertor generates an electric signal based on the light obtained by conversion by the scintillator. The thermal storage material is attached to the photoelectric convertor, and absorbs heat. To the rotating portion, the X-ray tube, the scintillator, the photoelectric convertor, and the thermal storage material are attached. The rotating mechanism rotates the rotating portion around a subject. The image generating circuitry generates an image based on the electric signal generated by the photoelectric convertor.

### CROSS-REFERENCE TO RELATED APPLICATIONS

This application is a continuation of PCT international application Ser. No. PCT/JP2014/060959 filed on Apr. 17, 2014 which designates the United States, incorporated herein by reference, and which claims the benefit of priority from Japanese Patent Application No. 2013-087765, filed on Apr. 18, 2013, the entire contents of which are incorporated herein by reference.

### FIELD

Embodiments described herein relate generally to an X-ray computed tomography (CT) apparatus.

### BACKGROUND

In recent years, X-ray CT apparatuses that use a photon counting detector are being developed. Unlike an integral detector used in a conventional X-ray CT apparatus, the photon counting detector counts light originated from X-rays that have passed through a subject body individually. Therefore, an X-ray CT apparatus that uses the photon counting detector can reconstruct an X-ray CT image with a high signal per noise (S/N) ratio. Moreover, the X-ray CT apparatus that uses a photon counting detector can divide one kind of an X-ray output into multiple energy components to form an image, and therefore, enables identification of a material using a difference in the K absorption edge. In photon counting detectors, for example, silicone photomultipliers (SiPM) are used as a photoelectric convertor.

However, because outputs of SiPM have remarkable temperature dependence, temperature control of SiPM is required to acquire stable outputs.

### BRIEF DESCRIPTION OF THE DRAWINGS

FIG. 1 is a block diagram of an X-ray CT apparatus of an embodiment;

FIG. 2 is a schematic diagram of the X-ray CT apparatus of the embodiment;

FIG. 3 is a schematic diagram of an inside of a base of the embodiment;

FIG. 4 is a schematic diagram 1 of an X-ray detector of the embodiment;

FIG. 5 is a schematic diagram 2 of an X-ray detector of the embodiment;

FIG. 6 is a schematic diagram 3 of an X-ray detector of the embodiment;

FIG. 7 is a schematic diagram of the X-ray detector and a cooling mechanism of the embodiment;

FIG. 8 is a flowchart indicating an operation of the embodiment;

FIG. 9 is a schematic diagram of an X-ray detector of a modification; and

FIG. 10 is a schematic diagram of a cooling mechanism of a modification.

### DETAILED DESCRIPTION

Embodiments of the present invention are explained below with reference to the accompanying drawings.

An X-ray computed tomography apparatus includes an X-ray tube, a scintillator, a photoelectric convertor, a thermal storage material, a rotating portion, a rotating mechanism, and image generating circuitry. The X-ray tube generates an X-ray. The scintillator converts the X-ray generated by the X-ray tube into light. The photoelectric convertor generates an electric signal based on the light obtained by conversion by the scintillator. The thermal storage material is attached to the photoelectric convertor, and absorbs heat. To the rotating portion, the X-ray tube, the scintillator, the photoelectric convertor, and the thermal storage material are attached. The rotating mechanism rotates the rotating portion around a subject. The image generating circuitry generates an image based on the electric signal generated by the photoelectric convertor.

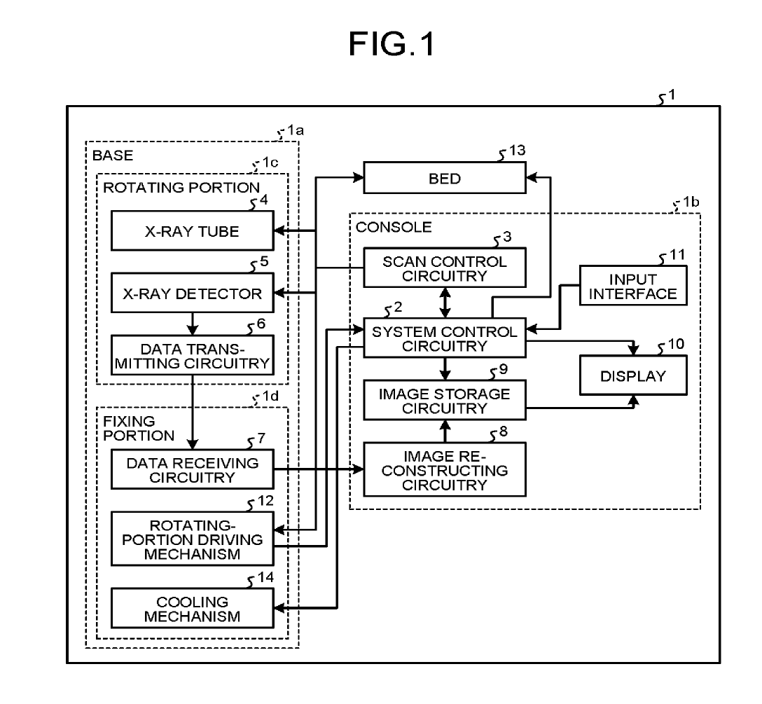

First, a configuration of an X-ray CT apparatus of a present embodiment is explained using either one of FIG. 1 to FIG. 7.

FIG. 1 is a block diagram of an X-ray CT apparatus 1 of the present embodiment.

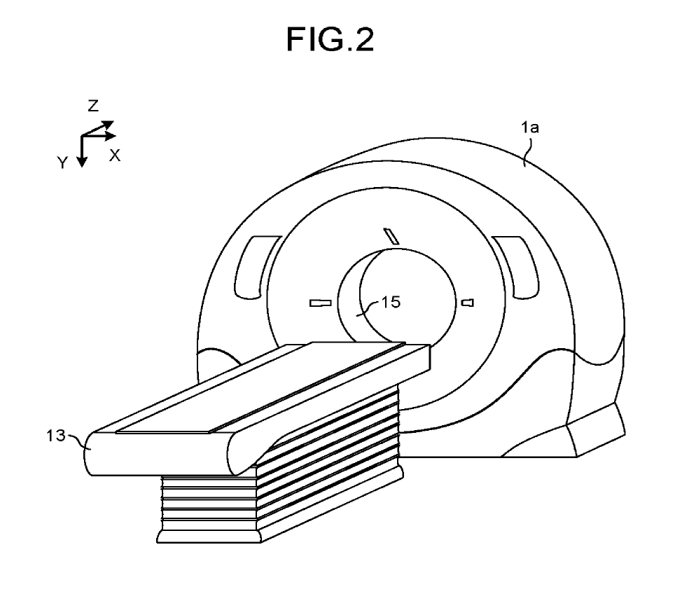

FIG. 2 is a schematic diagram of the X-ray CT apparatus 1 of the present embodiment.

As shown in FIG. 1 or FIG. 2, the X-ray CT apparatus 1 of the present embodiment includes a base 1a, a console 1b, and a bed 13. In the present embodiment, an axis that extends in a direction of a body axis of a subject that is laid on the bed 13 is a Z axis, an axis that extends in a vertical direction is a Y axis, and an axis that extends in a direction perpendicular to the Z axis and the Y axis is an X axis, and thus explanation is given below.

As shown in FIG. 1, the base 1a includes a rotating portion 1c, and a fixing portion 1d.

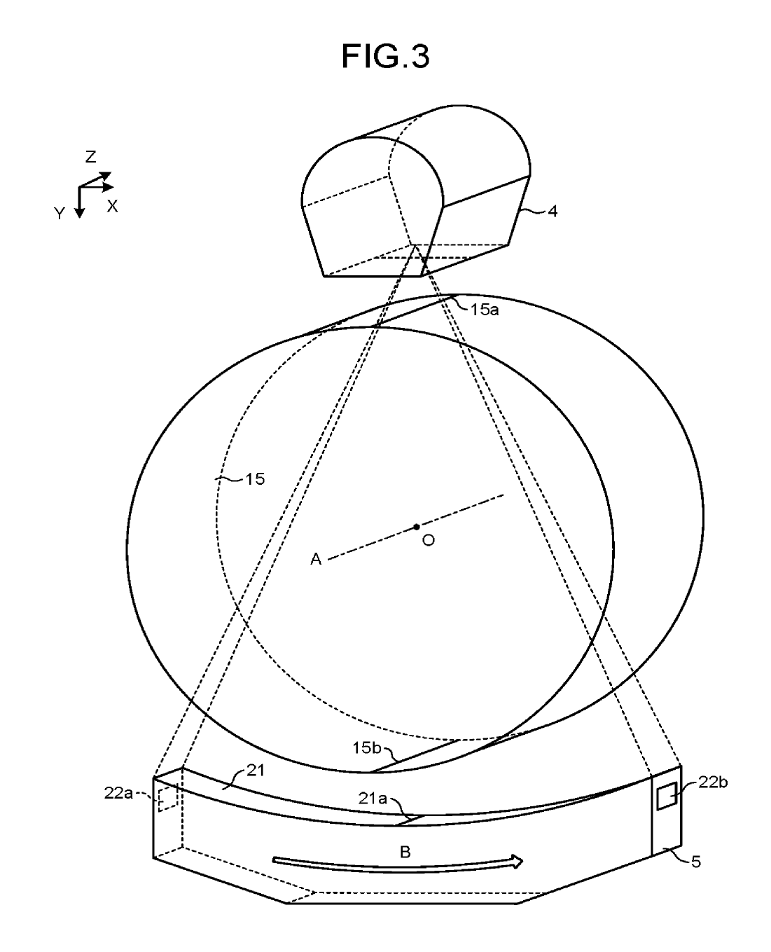

FIG. 3 is a schematic diagram of an inside of the base 1a.

As shown in FIG. 1, the rotating portion 1c includes an X-ray tube 4, an X-ray detector 5, and data transmitting circuitry 6. Furthermore, as shown in either one of FIG. 1 to FIG. 3, the fixing portion 1d includes data receiving circuitry 7, a rotating portion driving mechanism 12, a cooling mechanism 14, and an opening 15. The rotating portion 1c holds respective parts such that An X-ray that has been irradiated from the X-ray tube 4 and then passed through a subject enters a detecting surface 21 of the X-ray detector 5. The rotating portion 1c rotates about an axis (alternate long and short dashed line A) that passes through a center O of the opening 15 and is parallel to the Z axis, as a center based on an action of the rotating portion driving mechanism 12. The rotating portion 1c stops in such a state that the X-ray tube 4 is at a closest position to an opening top end 15a, and that a detecting surface center 21a in a curve direction B of the detecting surface 21 is at a closest position to an opening bottom end 15b, based on an action of the rotating portion driving mechanism 12.

As shown in FIG. 1, the console 1b includes system control circuitry 2, scan control circuitry 3, image reconstructing circuitry 8, image storage circuitry 9, a display 10, and an input interface 11.

The system control circuitry 2 causes the display 10 to display a predetermined input screen at predetermined timing. The system control circuitry 2 creates a scan plan according to an instruction of an operator input through the input interface 11. Details of the scan plan are not directly related to the present embodiment, and therefore, omitted. The system control circuitry 2 informs about the created scan plan to the scan control circuitry 3. The system control circuitry 2 informs the scan control circuitry 3 of a scan start when a scan start is instructed by an operator through the input interface 11. The system control circuitry 2 causes the display 10 to display an image that has been reconstructed by the image reconstructing circuitry 8 and stored in the image storage circuitry 9 according to an instruction of the operator received through the input interface 11. The system control circuitry 2 controls an action of the bed 13 according to an instruction of the operator received through the input interface 11. The system control circuitry 2 instructs the cooling mechanism 14 to start cooling when the X-ray CT apparatus 1 is activated. When informed of a start of rotation of the rotating portion 1c by the rotating portion driving mechanism 12, the system control circuitry 2 instructs the cooling mechanism 14 to stop cooling. When informed of a stop of rotation of the rotating portion 1c by the rotating portion driving mechanism 12, the system control circuitry 2 instructs the cooling mechanism 14 to start cooling according to an instruction of the operator received through the input interface 11.

When informed of a start of scan by the system control circuitry 2, the scan control circuitry 3 gives instructions to the X-ray tube 4, the X-ray detector 5, the rotating portion driving mechanism 12, and the bed 13. The scan control circuitry 3 instructs the X-ray tube 4 to start irradiation of X-rays at timing and intensity based on the scan plan that has been informed by the system control circuitry 2. The scan control circuitry 3 instructs the X-ray tube 4 to stop irradiation of X-rays at timing based on the scan plan that has been informed by the system control circuitry 2. The scan control circuitry 3 instructs the X-ray detector 5 to transmit a signal based on a detected X-ray to the data transmitting circuitry 6 at timing based on the scan plan. The scan control circuitry 3 instructs the rotating portion driving mechanism 12 to start rotation of the rotating portion 1c at timing based on the scan plan. The scan control circuitry 3 instructs the rotating portion driving mechanism 12 to stop rotation of the rotating portion 1c at timing based on the scan plan. The scan control circuitry 3 instructs the bed 13 to start moving the subject that is laid on the bed 13 to the direction of the Z axis at timing and speed based on the scan plan. The scan control circuitry 3 instructs the bed 13 to stop moving the subject that is laid on the bed 13 to the direction of the Z axis at timing based on the scan plan.

The X-ray tube 4 irradiates an X-ray based on an instruction of the scan control circuitry 3. The X-ray tube 4 stops irradiation of an X-ray based on an instruction of the scan control circuitry 3.

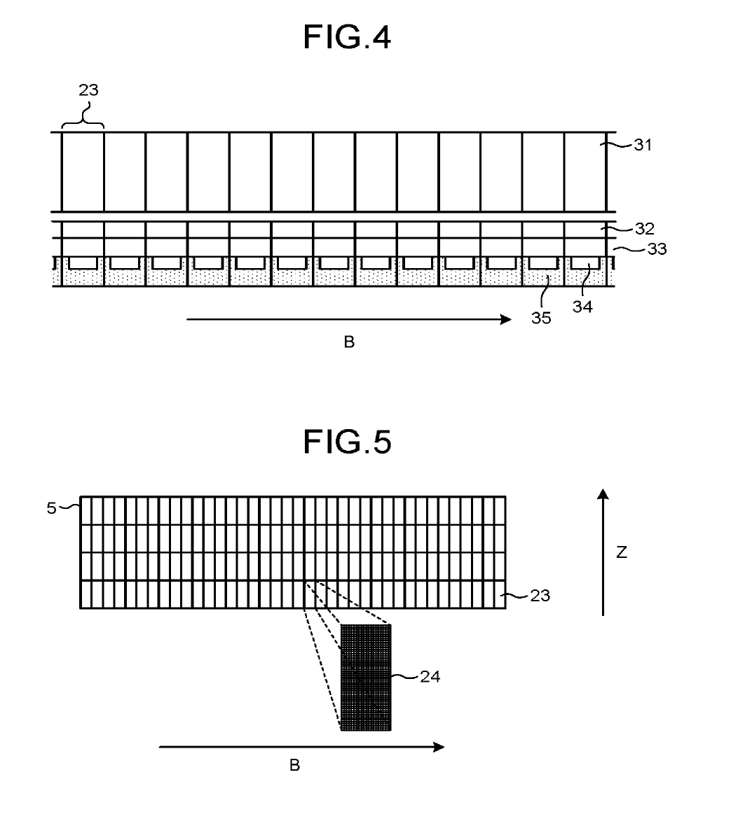

FIG. 4 is a schematic diagram illustrating an internal configuration of the X-ray detector 5 on a cross section parallel to an X-Y plane.

The X-ray detector 5 includes scintillators 31, SiPMs 32, substrates 33, complementary metal-oxide semiconductor (CMOS) circuits 34, and thermal storage portions 35.

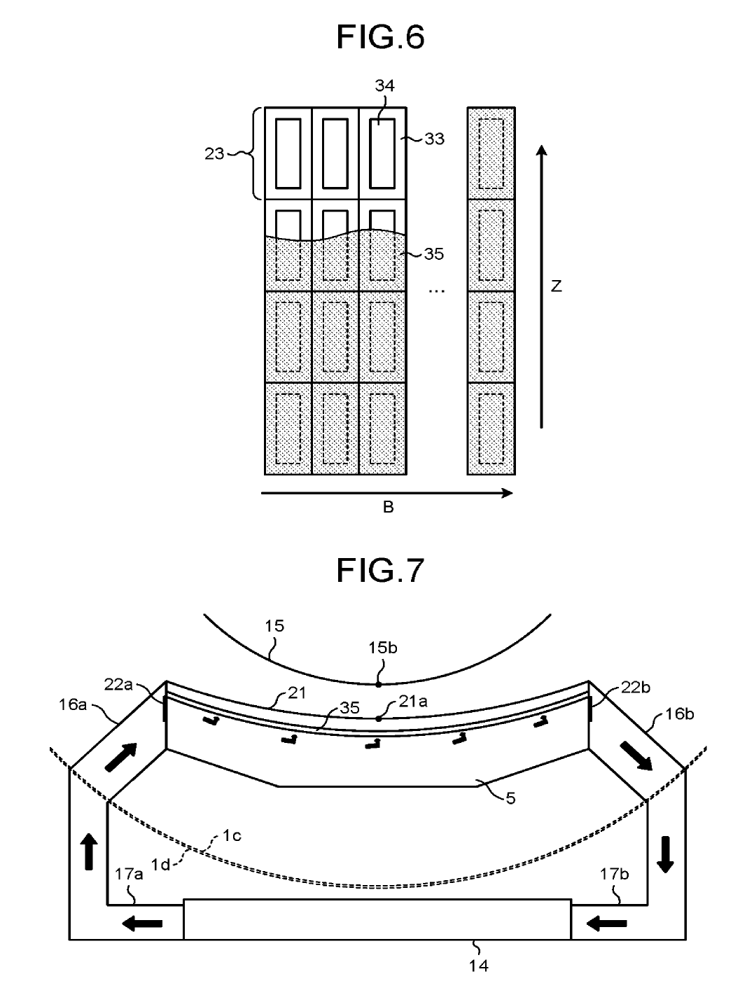

FIG. 5 and FIG. 6 are schematic diagrams illustrating an internal configuration of the X-ray detector 5 on a cross section parallel to the curve direction B and the Z axis.

In the present embodiment, for example, one unit of the scintillator 31 and one unit of the SiPM 32 form one device 24. Moreover, in the present embodiment, for example, one each of the substrate 33, the CMOS circuit 34, and the thermal storage portion 35, and multiple units of the devices 24 form one block 23. Assuming that the curve direction B and the direction of the Z axis are a row direction and a column direction, respectively, the X-ray detector 5 has 4 (rows)×38 (columns) blocks 23. Furthermore, the single block 23 includes, for example, as shown in FIG. 5, 64 (rows)×24 (columns) pieces of the devices 24. An X-ray that has been irradiated from the X-ray tube 4 and has passed through a subject laid on the bed 13 is collimated by each of the devices 24, and enters each of the devices 24.

The scintillator 31 generates light based on an incident X-ray.

The SiPM 32 is a photoelectric convertor, and generates an analog signal based on the light generated by the scintillator 31. The SiPM 32 is in contact with the substrate 33 as shown in FIG. 4.

The substrate 33 transmits, to the CMOS circuit 34, the analog signal generated by the SiPM 32 corresponding to each. The substrate 33 is in contact with the SiPM 32, the CMOS circuit 34, and the thermal storage portion 35 as shown in FIG. 4 or FIG. 6. The substrate 33 has a copper foil wide on a contact portion with the SiPM 32, and heat generated at the SiPM 32 is well conducted to the substrate 33. Furthermore, the substrate 33 has a copper foil wide on a contact portion with the thermal storage portion 35, and heat conducted from the SiPM 32 to the substrate 33 is well conducted to the thermal storage portion 35.

The CMOS circuit 34 converts the analog signal that is transmitted from the substrate 33 into a digital signal based on an instruction of the scan control circuitry 3, and transmits the digital signal to the data transmitting circuitry 6.

The thermal storage portion 35 is a temperature controller, and has a latent-heat storage material such as paraffin, calcium chloride hydrate, sodium sulfide hydrate, sodium thiosulfate hydrate, and sodium acetate hydrate, for example, in a container having high thermal conductivity. This latent-heat storage material absorbs heat generate at the SiPM 32 and conducted through the substrate 33. As described above, in the present embodiment, heat generated at the SiPM 32 is well conducted to the thermal storage portion 35 through the substrate 33, and therefore, the temperature of the latent-heat storage material and the temperature of the SiPM 32 are to be equal to each other. In the following, for simplicity's sake, for example, explanation is given assuming that the temperature of the latent-heat storage material and the temperature of the SiPM 32 are equal, ignoring a difference in specific heat between the respective parts.

For example, when the temperature of the latent-heat storage material and the temperature of the SiPM 32 are lower than the melting temperature of the latent-heat storage material, the temperature of the latent-heat storage material first increases to the melting temperature based on heat generated at the SiPM 32 and conducted through the substrate 33. When the temperature of the latent-heat storage material reaches the melting temperature, the latent-heat storage material starts accumulating the heat that is generated at the SiPM 32 and is conducted through the substrate 33. The temperature of the latent-heat storage material is maintained constant as long as the amount of accumulated heat does not exceed the heat of fusion of the latent-heat storage material. Therefore, even if heat is generated at the SiPM 32 at this time, the heat is conducted to the latent-heat storage material through the substrate 33 to be accumulated, and therefore, the temperature of the SiPM 32 is maintained constant. When heat is further generated at the SiPM 32 and the amount of heat accumulated in the latent-heat storage material finally exceeds the heat of fusion of the latent-heat storage material, the temperature of the latent-heat storage material increases, and the temperature of the SiPM 32 also increases. For example, when the latent-heat storage material is paraffin expressed by a composition formula below, the melting temperature is approximately 28° C., and the heat of fusion is approximately 240 kJ/kg.

C₁₈H₃₈ [Formula 1]

The data transmitting circuitry 6 includes, for example, an optical communication device, and converts a digital signal that is received from the CMOS circuit 34 into optical data, and transmits the optical data to the data receiving circuitry 7 of the fixing portion 1d by using the optical communication device.

The data receiving circuitry 7 generates projection data based on the optical data received from the data transmitting circuitry 6, and transmits the projection data to the image reconstructing circuitry 8.

The image reconstructing circuitry 8 reconstructs an image based on the projection data received from the data receiving circuitry 7. The image reconstructing circuitry 8 transmits the reconstructed image to the image storage circuitry 9.

The image storage circuitry 9 stores the image received from the image reconstructing circuitry 8.

The display 10 displays the image stored in the image storage circuitry 9 according to an instruction of the system control circuitry 2. The display 10 displays a predetermined input screen according to an instruction of the system control circuitry 2.

The input interface 11 includes, for example, a mouse and a keyboard, and gives an instruction based on an input made by an operator using these components to the system control circuitry 2.

The rotating portion driving mechanism 12 rotates the rotating portion 1c based on an instruction of the scan control circuitry 3. The rotating portion driving mechanism 12 stops rotation of the rotating portion 1c based on an instruction of the scan control circuitry 3. When stopping rotation of the rotating portion 1c, the rotating portion driving mechanism 12 brings into the state that the X-ray tube 4 is at the closest position to the opening top end 15a, and that the detecting surface center 21a in the curve direction B of the detecting surface 21 is at the closest position to the opening bottom end 15b, as described above. When starting rotation of the rotating portion 1c, the rotating portion driving mechanism 12 informs the system control circuitry 2 that rotation of the rotating portion is is to be started. When rotation of the rotating portion 1c is stopped, the rotating portion driving mechanism 12 informs the system control circuitry 2 that rotation of the rotating portion 1c has stopped.

The bed 13 moves a subject that is laid thereon to the directions of the X axis, the Y axis, and the Z axis according to an instruction of the system control circuitry 2. The bed 13 moves the subject laid thereon to the directions of the X axis, the Y axis, and the Z axis based on an instruction of the scan control circuitry 3. The bed 13 stops moving the subject laid thereon based on an instruction of the scan control circuitry 3.

The cooling mechanism 14 is a cooling portion to cool the thermal storage portion 35, and generates cold air according to an instruction of the system control circuitry 2. The cooling mechanism 14 stops generated cold air according to an instruction of the system control circuitry 2.

FIG. 7 is a schematic diagram of the X-ray detector 5 and the cooling mechanism 14 of the present embodiment.

The rotating portion 1c includes a duct 16a and a duct 16b as shown in FIG. 7, in addition to the components described above. The fixing portion 1d includes a duct 17a and a duct 17b as shown in FIG. 7, in addition to the components described above. The X-ray detector 5 includes a vent 22a and a vent 22b as shown in FIG. 3 and FIG. 7, in addition to the components described above. The cold air generated by the cooling mechanism 14 is sent so as to circulate in order of the duct 17a, the duct 16a, the vent 22a, an inside of the X-ray detector 5, the vent 22b, the duct 16b, and then the duct 17b, for example. As shown in FIG. 7, the duct 16a, the duct 17a, the duct 16b, and the duct 17b are connected during the rotating portion 1c is stopped, that is, in a state in which the detecting surface center 21a in the curve direction B of the detecting surface 21 is at the closest position to the opening bottom end 15b. In the present embodiment, the temperature of the cold air generated by the cooling mechanism 14 is, for example, the melting temperature of the latent-heat storage material, and it is configured such that the cold air removes heat accumulated in the latent-heat storage material but does not make the temperature of the latent-heat storage material lower than the melting temperature.

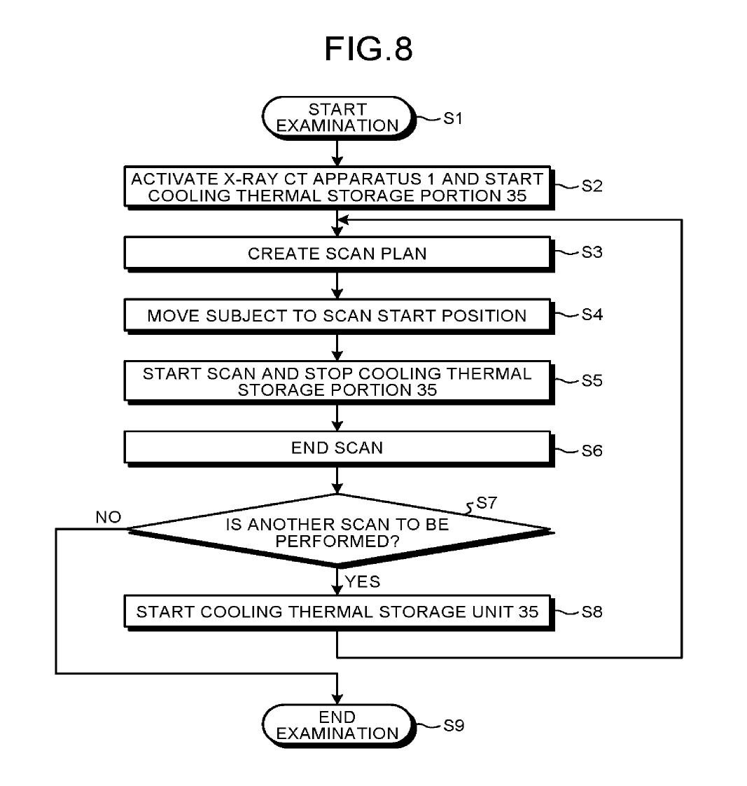

Next, an operation of the present embodiment is explained using a flowchart in FIG. 8.

At step S1, an examination is started.

At step S2, an operator activates the X-ray CT apparatus 1. When the X-ray CT apparatus 1 is activated, the system control circuitry 2 instructs the cooling mechanism 14 to start cooling. The cooling mechanism 14 generates cold air according to the instruction of the system control circuitry 2. The cold air generated by the cooling mechanism 14 is sent so as to circulate in order of the Duct 17a, the duct 16a, the vent 22a, the inside of the X-ray detector 5, the vent 22b, the duct 16b, and then the duct 17b, and cools the thermal storage portion 35 to the melting temperature of the latent-heat storage material. Moreover, the system control circuitry 2 causes the display 10 to display an input screen to create a scan plan.

At step S3, the operator refers to the input screen to create a scan plan displayed on the display 10, and makes an input through the input interface 11. The system control circuitry 2 creates a scan plan according to an instruction of the operator input through the input interface 11. The system control circuitry 2 informs about the created scan plan to the scan control circuitry 3.

At step S4, the operator lays a subject on the bed 13. Furthermore, the operator makes an input to move the laid subject to a scan start position into the input interface 11. The system control circuitry 2 controls the action of the bed 13 according to the instruction of the operator input through the input interface 11. The bed 13 moves the position of the subject to the scan start position according to a control of the system control circuitry 2. When the subject is moved to the scan start position by the action of the bed 13, the operator makes an input to instruct a scan start to the system control circuitry 2, into the input interface 11.

At step S5, when a scan start is instructed by the operator through the input interface 11, the system control circuitry 2 informs the scan control circuitry 3 of the scan start. When the scan start is informed by the system control circuitry 2, the scan control circuitry 3 gives instructions to the X-ray tube 4, the X-ray detector 5, the rotating portion driving mechanism 12, and the bed 13. The scan control circuitry 3 starts irradiation of an X-ray at timing and intensity based on the scan plan that has been informed by the system control circuitry 2, and instructs the X-ray tube 4 to stop irradiation of the X-ray at timing based on the scan plan. The scan control circuitry 3 instructs the X-ray detector 5 to transmit a signal based on a detected X-ray to the data transmitting circuitry 6 at timing based on the scan plan. The scan control circuitry 3 instructs the rotating portion driving mechanism 12 to start rotation of the rotating portion 1c at timing based on the scan plan, and to stop rotation of the rotating portion 1c at timing based on the scan plan. The scan control circuitry 3 instructs the bed 13 to start moving the subject laid thereon in the direction of the Z axis at timing and speed based on the scan plan, and to stop moving the subject laid thereon in the direction of the Z axis at timing based on the scan plan.

When the instructions by the scan control circuitry 3 are given, the X-ray tube 4, the X-ray detector 5, the rotating portion driving mechanism 12, and the bed 13 performs various operations based on the instructions of the scan control circuitry 3.

The rotating portion driving mechanism 12 rotates the rotating portion 1c based on the instruction of the scan control circuitry 3. At this time, the rotating portion driving mechanism 12 informs the system control circuitry 2 that rotation of the rotating portion 1c is to be started. When informed that rotation of the rotating portion 1c is to be started from the rotating portion driving mechanism 12, the system control circuitry 2 instructs the cooling mechanism 14 to stop cooling. The cooling mechanism 14 stops generated cold air according to the instruction of the system control circuitry 2.

The X-ray tube 4 irradiates an X-ray based on the instruction of the scan control circuitry 3. The scintillator 31 generates light based on an X-ray that has passed through the subject laid on the bed 13 and has entered therein. The SiPM 32 generates an analog signal based on the light generated by the scintillator 31. The substrate 33 transmits, the CMOS circuit 34, the analog signal generated by the SiPM 32 corresponding to each. The CMOS circuit 34 converts the analog signal transmitted from the substrate 33 to a digital signal based on an instruction of the scan control circuitry 3, and transmits the digital signal to the data transmitting circuitry 6. The data transmitting circuitry 6 converts the digital signal received from the CMOS circuit 34 into optical data, and transmits the optical data to the data receiving circuitry 7 of the fixing portion 1d by using the optical communication device. The data receiving circuitry 7 generates projection data based on the optical data received from the data transmitting circuitry 6, and transmits the projection data to the image reconstructing circuitry 8. The image reconstructing circuitry 8 reconstructs an image based on the projection data received from the data receiving circuitry 7. The image reconstructing circuitry 8 transmits the reconstructed image to the image storage circuitry 9. The image storage circuitry 9 stores the image received from the image reconstructing circuitry 8.

The bed 13 moves the subject laid thereon based on the instruction of the scan control circuitry 3.

At step S6, when a scan based on the scan plan created at step S3 is finished, the X-ray tube 4, the rotating portion driving mechanism 12, and the bed 13 performs various operations based on the instructions of the scan control circuitry 3 at step S5.

The X-ray tube 4 stops irradiation of an X-ray based on the instruction of the scan control circuitry 3 at step S5.

The bed 13 stops moving the subject based on the instruction of the scan control circuitry 3 at step S5.

The rotating portion driving mechanism 12 stops rotation of the rotating portion 1c based on the instruction of the scan control circuitry 3 at step S5. When stopping rotation of the rotating portion 1c, the rotating portion driving mechanism 12 brings into the state that the X-ray tube 4 is at the closest position to the opening top end 15a, and that the detecting surface center 21a in the curve direction B of the detecting surface 21 is at the closest position to the opening bottom end 15b, as described above. When rotation of the rotating portion 1c is stopped, the rotating portion driving mechanism 12 informs the system control circuitry 2 that rotation of the rotating portion 1c has stopped.

At step S7, the system control circuitry 2 causes the display 10 to display a selecting screen to select whether to perform another scan. When another scan is to be performed (step S7: YES), the operator selects an option to perform another scan through the input interface 11. In this case, the flow shifts to step S8. On the other hand, when another scan is not to be performed (step S7: NO), the operator selects an option not to perform another scan through the input interface 11. In this case, the flow shifts to step S9.

At step S8, the system control circuitry 2 instructs the cooling mechanism 14 to start cooling. The cooling mechanism 14 generates cold air according to the instruction of the system control circuitry 2. The cold air generated by the cooling mechanism 14 is sent to be circulated in order of the duct 17a, the duct 16a, the vent 22a, the inside of the X-ray detector 5, the vent 22b, the duct 16b, and then the duct 17b, to cool the thermal storage portion 35 to the melting temperature of the latent-heat storage material. Moreover, the system control circuitry 2 causes the display 10 to display an input screen to create a scan plan, and the flow shifts to step S3.

At step S9, the examination is ended.

As explained above, in the X-ray CT apparatus 1 of the present embodiment, heat generated by the SiPM 32 at the time of scanning is absorbed by the thermal storage portion 35, and the temperature of the SiPM 32 is maintained at the melting temperature of the latent-heat storage material. Moreover, in the X-ray CT apparatus 1 of the present embodiment, when the rotating portion 1c is stopped, the thermal storage portion 35 is cooled at the melting temperature of the latent-heat storage material to remove heat accumulated in the latent-heat storage material included in the thermal storage portion 35. This enables stable output of the SiPM 32 that is remarkably temperature dependent, and to reconstruct a highly reliable X-ray CT image. Furthermore, in the X-ray CT apparatus 1 of the present embodiment, complicated temperature controller or cooling portion are not required to be equipped in the rotating portion 1c, and increase in size of the rotating portion 1c can be avoided.

Although the present embodiment has been explained with paraffin having the melting temperature of approximately 28° C. and the heat of fusion of approximately 240 kJ/kg as a specific example of the latent-heat storage material, a latent-heat storage material having a lower melting temperature and higher heat of fusion can be used in the thermal storage portion 35. When a latent-heat storage material having lower melting temperature is used in the thermal storage portion 35, the S/N ratio of the analog signal generated by the SiPM 32 can be lowered. Moreover, when a latent-heat storage material having higher heat of fusion is used in the thermal storage portion 35, the temperature of the SiPM 32 can be maintained further stable.

Although, in the present embodiment, a case of controlling the temperature of the SiPM 32 and the thermal storage portion 35 by fixing the temperature of cold air that is generated by the cooling mechanism 14 to a predetermined temperature has been explained, for example, the temperature sensor can provided in the SiPM 32 or the thermal storage portion 35, and the temperature of the cold air generated by the cooling mechanism 14 can be changed based on the temperature of the SiPM 32 or the thermal storage portion 35 detected by this temperature sensor. Furthermore, when the temperature of the thermal storage portion 35 detected by this temperature sensor rises and exceeds the melting temperature of the latent-heat storage material, a scan may be suspended and the thermal storage portion 35 may be cooled.

Although, in the present embodiment, a case in which the temperature of the thermal storage portion 35 is maintained at the melting temperature of the latent-heat storage material to maintain the temperature of the SiPM 32 indirectly has been explained, the temperature of the SiPM 32 can be maintained at a temperature lower than the melting temperature of the latent-heat storage material if, for example, a Peltier device, a temperature sensor, and a temperature controller are used.

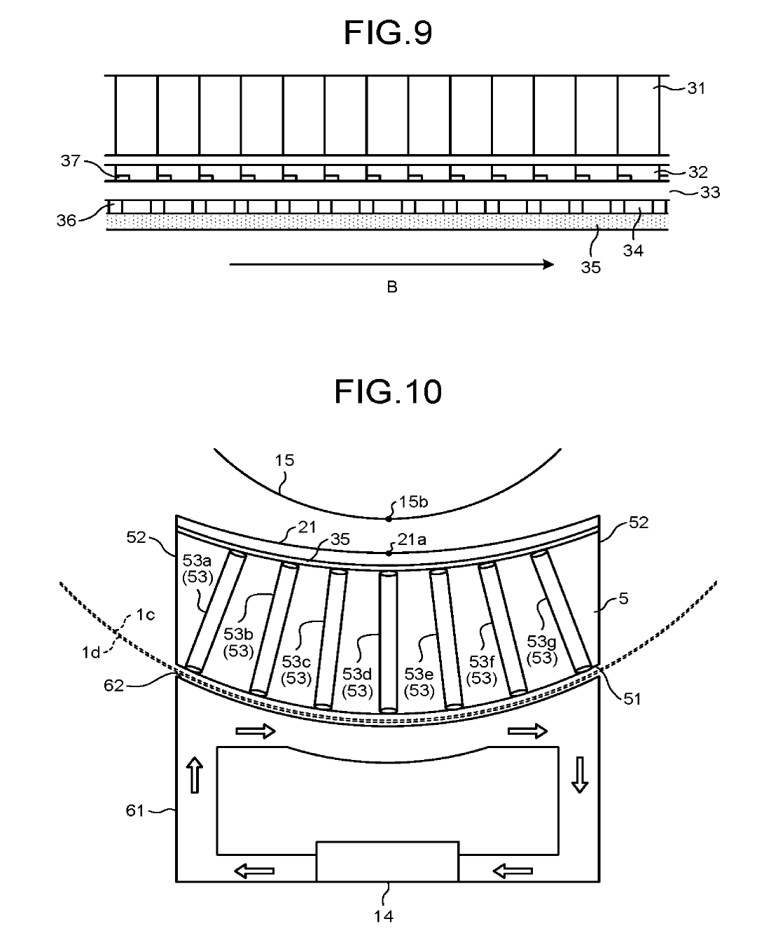

FIG. 9 is a schematic diagram of an internal configuration of the X-ray detector 5 of a modification.

In this modification, the X-ray detector 5 includes a Peltier device 36 between the substrate 33 and the thermal storage portion 35, and a temperature sensor 37 between the SiPM 32 and the substrate 33. The Peltier device 36 has an endothermic surface and an exothermic surface, and the endothermic surface and the exothermic surface are in contact with the substrate 33 and the thermal storage portion 35, respectively. The Peltier device 36 is connected to a temperature controller not shown, and absorbs heat from the endothermic surface and dissipates heat from the exothermic surface when an electric current is applied by the temperature controller. The temperature sensor 37 detects the temperature of the SiPM 32, and informs the temperature of the SiPM 32 to the temperature controller. The temperature controller applies an electric current to the Peltier device 36 so that the temperature of the SiPM 32 informed by the temperature sensor 37 is constant. In this modification, for example, heat dissipated from the Peltier device 36 to the thermal storage portion 35 is absorbed by the thermal storage portion 35. The thermal storage portion 35 is cooled while the rotating portion 1c is stopped, and thus the heat accumulated in the thermal storage portion 35 is removed. In this case, because it is not essential to maintain the temperature of the thermal storage portion 35 constant, the thermal storage portion 35 is not required to include the latent-heat storage material. As a substitute for the latent-heat storage material, for example, a member having large thermal capacity, and the like can be applied.

Although a case in which the temperature of the SiPM 32 and the temperature of the thermal storage portion 35 are equal to each other has been explained in the present embodiment for simplicity's sake, in an actual state, because there is a difference in specific heat and the like therebetween, there is a difference between the temperature of the SiPM 32 and the temperature of the thermal storage portion 35. In this case also, the temperature of the SiPM 32 is maintained substantially constant due to the melting temperature of the latent-heat storage material, and therefore, a similar effect as the effect explained in the present embodiment can be obtained. Moreover, although a case in which if the cooling mechanism 14 blows cold air at a temperature equal to the melting temperature of the latent-heat storage material of the thermal storage portion 35, the temperature of the thermal storage portion 35 is maintained at the melting temperature has been explained in the present embodiment for simplicity's sake, in an actual state, because there is a difference in specific heat and the like therebetween, there is a difference between the temperature of the cold air and the temperature of the thermal storage portion 35 cooled thereby. In this case, for example, by setting the temperature of the cold air to a lower temperature than the melting temperature such that the temperature of the thermal storage portion 35 is maintained at the melting temperature, a similar effect as the effect of the present embodiment can be obtained.

Although a case in which the cooling mechanism 14 generates cold air, and the thermal storage portion 35 is cooled by the cold air has been explained in the present embodiment, another cooling means such as a heat pipe may be used to cool the thermal storage portion 35.

Alternatively, it may be configured such that for example, heat accumulated in the thermal storage portion 35 is moved to a predetermined region of the rotating portion 1c by a heat pipe, and the cooling mechanism 14 cools the predetermined region of the rotating portion 1c. The predetermined region of the rotating portion 1c is, for example, a part of region that is positioned near a bottom surface of the rotating portion 1c when the rotating portion 1c is stopped.

FIG. 10 is a schematic diagram of the cooling mechanism of a modification. As shown in FIG. 10, in the rotating portion 1c, a bottom surface 51 and a side surface 52 of the X-ray detector 5 are structured with different materials. For example, the bottom surface 51 of the X-ray detector 5 is a material having high thermal conductivity, and the side surface 52 of the X-ray detector 5 is a material having low thermal conductivity. Moreover, as shown in FIG. 10, the X-ray detector 5 further includes heat pipes 53a to 53g in addition to the components described above. When the heat pipes 53a to 53g are not distinguished, it is referred to as heat pipe 53.

Each of the heat pipes 53 is connected to the thermal storage portion 35 and the bottom surface 51 of the X-ray detector 5. Therefore, each of the heat pipes 53 transfers heat accumulated in the thermal storage portion 35 to the bottom surface 51 of the X-ray detector 5. Having high thermal conductivity, the bottom surface 51 of the X-ray detector 5 accumulates the heat transferred by each of the heat pipes 53. Because the side surface 52 of the X-ray detector 5 has low thermal conductivity, the side surface 52 does not accumulate the heat accumulated in the thermal storage portion 35.

Furthermore, the fixing portion 1d includes a duct 61 as shown in FIG. 10 in addition to the components described above. This duct 61 is connected to the cooling mechanism 14 so as to extend from one end of the cooling mechanism 14, and return to the other end of the cooling mechanism 14. As shown in FIG. 10, a part of a surface 62 of the duct 61 is brought into intimate contact with the bottom surface 51 of the X-ray detector 5 when the rotating portion 1c is stopped, that is, in the state that the detecting surface center 21a in the curve direction B of the detecting surface 21 is at the closest position to the opening bottom end 15b. At the time of stopping rotation, the rotating portion driving mechanism 12 rotates the rotating portion 1c by such an angle that the bottom surface 51 of the X-ray detector 5 and a part of the surface 62 of the duct 61 are positioned close to each other in the rotating portion 1c. In such a state, cold air generated by the cooling mechanism 14 is blown so as to circulate in order indicated by arrows in the duct 61. Thus, heat accumulated at the bottom surface 51 of the X-ray detector 5 is cooled by the cooling mechanism 14. In other words, the cooling mechanism 14 cools a predetermined region of the rotating portion 1c when rotation of the rotating portion 1c is stopped. Although it has been explained that the cooling mechanism 14 blows cold air inside the duct 61, embodiments are not limited thereto. For example, the cooling mechanism 14 can circulate cooling water in the duct 61. It can be referred to as a cooling portion including the cooling mechanism 14 and the duct 61.

Furthermore, when the rotating portion 1c can be tilted, the rotating portion 1c can be rotated in a tilted manner by a predetermined tilting angle. In such a case, after rotation of the rotating portion 1c is stopped, the rotating portion 1c is returned to a state before tilted by the predetermined tilting angle. Thus, the thermal storage portion 35 can be cooled by the cooling mechanism 14. For example, as shown in FIG. 7, the duct 16a, the duct 17a, the duct 16b, and the duct 17b are connected during the rotating portion 1c is stopped, that is, in the state that the detecting surface center 21a in the curve direction B of the detecting surface 21 is at the closest position to the opening bottom end 15b. Thus, heat accumulated in the thermal storage portion 35 can be cooled by the cooling mechanism 14. For example, as shown in FIG. 10, the part of the surface 62 of the duct 61 is brought into intimate contact with the bottom surface 51 of the X-ray detector 5 during the rotating portion 1c is stopped, that is, in the state that the detecting surface center 21a in the curve direction B of the detecting surface 21 is at the closest position to the opening bottom end 15b.

The Peltier device 36 explained in the embodiment described above can be controlled such that an electric current can be applied in a unit of an area that is obtained by dividing the SiPM 32 into multiple areas. In such a case, when there is a difference between the temperature of one area of the SiPM 32 and the temperature of another area of the SiPM 32, the temperature controller applies a different electric current to the Peltier device 36 according to the temperature, for the SiPM 32 arranged in each of the areas. By thus configuring, even when temperature unevenness occurs in the SiPM 32, the SiPM 32 can be appropriately cooled. Moreover, the temperature controller may control the respective Peltier devices 36 individually, or may control all of the Peltier devices at the same time.

Although a case in which execution of scan is prioritized and cooling is suspended irrespective of the cooling time of the thermal storage portion 35 has been explained in the present embodiment, it can be configured such that, for example, the predetermined time is set so as to ensure that the thermal storage portion 35 is cooled, and a scan is executed after this predetermined time has elapsed. In this case, it is possible, for example, to remove heat accumulate in the thermal storage portion 35 for only a predetermined amount each time, and the output of the SiPM 32 can be further stabled.

Although a case in which a photoelectric convertor is the SiPM has been explained in the present embodiment, the present embodiment is applicable as long as, for example, a photoelectric convertor having remarkable temperature dependence is used.

According to at least one of the embodiments explained above, the temperature of a photoelectric convertor can be controlled to be near a predetermined temperature.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography (CT) apparatus comprising:
an X-ray tube configured to generate an X-ray;
a scintillator configured to convert the X-ray generated by the X-tube into light;
a photoelectric convertor configured to generate an electric signal based on the light obtained by conversion by the scintillator;
a thermal storage material configured to be attached to the photoelectric convertor, and that absorbs heat;
a rotating portion to which the X-ray tube, the scintillator, the photoelectric convertor, and the thermals storage material are attached;
a rotating mechanism configured to rotate the rotating portion around a subject; and
image generating circuitry configured to generate an image based on the electric signal generated by the photoelectric convertor.

2. The X-ray CT apparatus according to claim 1 further comprising:
a cooling portion configured to cool the thermals storage material; and
a base on which the cooling portion is arranged, and that supports the rotating portion and is set on a surface of a floor, wherein
the rotating mechanism rotates, when rotation is to be stopped, the rotating portion by such an angle that the thermal storage material and the cooling portion are positioned close to each other, and
the cooling portion cools the thermal storage material when rotation of the rotating portion is stopped.

3. The X-ray CT apparatus according to claim 1, wherein
the thermal storage material absorbs heat that is generated at the photoelectric convertor, and is a latent-heat storage material that maintains temperature of the photoelectric convertor at a melting temperature of the thermal storage material.

4. The X-ray CT apparatus according to claim 2, wherein
the thermal storage material absorbs heat that is generated at the photoelectric convertor, and is a latent-heat storage material that maintains temperature of the photoelectric convertor at a melting temperature of the thermal storage material.

5. The X-ray CT apparatus according to claim 2, wherein
the cooling portion controls an amount of heat of the thermal storage material to be cooled so that the temperature of the thermal storage material is maintained at a melting temperature.

6. The X-ray CT apparatus according to claim 3, wherein
the latent-heat storage material includes at least one of paraffin, calcium chloride hydrate, sodium sulfide hydrate, sodium thiosulfate hydrate, and sodium acetate hydrate.

7. The X-ray CT apparatus according to claim 4, wherein
the latent-heat storage material includes at least one of paraffin, calcium chloride hydrate, sodium sulfide hydrate, sodium thiosulfate hydrate, and sodium acetate hydrate.

8. The X-ray CT apparatus according to claim 1 further comprising
a heat conducting mechanism configured to conduct heat generated by the photoelectric convertor to the thermal storage material, wherein
the heat conducting mechanism includes
a Peltier device that has an endothermic surface and an exothermic surface, and that absorbs heat generated at the photoelectric convertor by the endothermic surface to dissipate to the thermal storage material that is connected to the exothermic surface when an electric current is applied;
a temperature sensor that measures temperature of the photoelectric convertor; and
a temperature controller that applies an electric current to the Peltier device based on the temperature measured by the temperature sensor.

9. The X-ray CT apparatus according to claim 2, comprising
a temperature sensor configured to measure temperature of the photoelectric convertor, wherein
the cooling portion cools the thermal storage material based on the temperature of the photoelectric convertor measured by the temperature sensor.

10. The X-ray CT apparatus according to claim 2 comprising
a temperature sensor configured to measure temperature of the photoelectric convertor, wherein
the X-ray tube suspends generation of an X-ray when the temperature of the photoelectric convertor measured by the temperature sensor exceeds a predetermined value;
the rotating mechanism stops rotation of the rotating portion when the temperature of the photoelectric convertor measured by the temperature sensor exceeds the predetermined value;
the cooling portion starts cooling when the temperature of the photoelectric convertor measured by the temperature sensor exceeds the predetermined value.

11. The X-ray CT apparatus according to claim 1, wherein
the photoelectric convertor is a silicone photomultiplier.

12. The X-ray CT apparatus according to claim 1 further comprising:
a heat conductor configured to transfer heat accumulated in the thermal storage material to a predetermined region of the rotating portion; and
a base on which the cooling portion is arranged, and that supports the rotating portion and is set on a surface of a floor, wherein
the rotating mechanism rotates, when rotation is to be stopped, the rotating portion by such an angle that a predetermined region of the rotating portion and the cooling portion are positioned close to each other, and
the cooling portion cools the predetermined region of the rotating portion when rotation of the rotating portion is stopped.
